(19) United States
(12) Patent Application Publication
(10) Pub. No.: US 2004/0219066 A1
(43) Pub. Date: Nov. 4, 2004

(54) Apparatus used in identification, sorting and collection methods using magnetic microspheres and magnetic microsphere kits

(76) Inventors: Robert H. Kraus JR., Los Alamos, NM (US); Feng Zhou, Los Alamos, NM (US); John Nolan, Santa Fe, NM (US)

Correspondence Address: UNIVERSITY OF CALIFORNIA, LOS ALAMOS NATIONAL LABORATORY, P.O. BOX 1663, MS A187, LOS ALAMOS, NM, 87545, US

(21) Appl. No.: 10/851,019

(22) Filed: May 21, 2004

Related U.S. Application Data
(62) Division of application No. 09/995,302, filed on Nov. 27, 2001, now abandoned.

(51) Int. Cl. G01N 33/00

(52) U.S. Cl. 422/72

(57) ABSTRACT

The present invention provides a particle identification apparatus including a flow cell for passage of fluid containing a population of labeled magnetic microspheres in a stream, the magnetic microspheres having a label providing a detectable property to the magnetic microspheres, and, a magnetic measurement system, positioned adjacent to the flow cell, for measuring a magnetic moment on each labeled magnetic microsphere as it passes by the magnetic measurement system. The present invention also provides a particle sorting apparatus including a chamber having an inlet for a fluid suspension of a population of magnetic microspheres to be sorted, a magnetic field generator that produces a field gradient across the chamber for producing a force on the magnetic microspheres within the fluid suspension, a series of collection bins positioned within the chamber for receiving magnetic microspheres with distinctly different magnetic moments as a result of movement of the magnetic microspheres resulting from the force produced on the magnetic microspheres within the fluid suspension by magnetic field gradient; and, an outlet for fluid flow. The present invention also provides a kit for sorting and identifying a material within a sample, the kit including a population of magnetic microspheres each having a distinctly measurable magnetic moment, with each individual magnetic microsphere also having one or more receptor agents attached thereto, and, a population of non-magnetic microspheres, with each individual non-magnetic microsphere also having one or more receptor agents attached thereto. The present invention also provides a kit for sorting and identifying a material within a sample, the kit including at least two populations of magnetic microspheres each population having a distinctly different measurable magnetic moment.

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates generally to a method for identification and sorting of molecules, especially biomolecules, using receptor labeled magnetic microspheres.

### BACKGROUND OF THE INVENTION

Biomedical research has evolved significantly over the past several years, with the large-scale screening of whole genomes complementing focused studies on a few genes or proteins. This evolution has encompassed applications ranging from functional analysis of unknown genes to identification of disease-related genes, screening in drug discovery and clinical diagnostics. There has been a concurrent surge in technology development to facilitate large-scale biological analysis. In general, these technologies have two components, the assay chemistry and the detection platform. Perhaps the best publicized detection platform of recent years is the flat microarray. Configured as “DNA chips,” these flat microarrays offer the promise of whole genome analysis of single samples. Each element or “spot” on a flat surface array contains a target-specific receptor, for example a DNA molecule to detect a specific DNA sequence, and the signal originating from that element reports the presence of a target molecule. A related detection platform that is proving to be compatible with a range of assay chemistries in a high-throughput format is the use of encoded microparticle in combination with flow-based analysis cytometry, also known as Suspension Array Technology (SAT).

Suspension array technology employs fluorescence-encoded microspheres as array elements that bear specific receptor molecules. In SAT, microspheres having distinct optical properties, for example light scatter or fluorescence from an internal dye, are employed as solid supports for a variety of molecular analyses. By careful adjustment of these intrinsic optical properties, it is possible to prepare arrays of microspheres in which individual microsphere subsets can be identified and used to perform multiplexed analysis. Conceptually, microsphere arrays are similar to flat-surface microarrays, with distinct quanta of intrinsic optical parameters substituting for physical location on a surface. While fluorescent- or optically-encoded microspheres have improved the flexibility of array-based analysis, that approach faces limitations in both the preparation and use of the microspheres. For example, the ability to reproducibly dye microspheres is problematic such that lot-to-lot variations in microparticles can be a problem. Moreover, the use of fluorescent dyes to encode the microparticles limits the number of analytical measurements, which also employ fluorescence detection, that can be made. An encoding method that did not require, but that was compatible with, fluorescence detection would be desirable.

The analysis of single nucleotide polymorphisms (SNPs), provides a useful example of the types of analysis that can be performed. The human genome project has shown that the DNA sequence from any two individuals is about 99.9% identical, and that the phenotypic differences between individuals are conferred largely by the 0.1% of the sequence that is different. The vast majority of this sequence variation is in the form of single nucleotide polymorphisms (or SNPs), sites in the genome where a single base varies between chromosomes in the same individual or between different individuals.

As genetic markers, SNPs have great potential for use in disease diagnostics and the discovery of new drugs. Major pharmaceutical companies and academic genome centers are involved in a major effort to discover and map SNPs. Unfortunately, conventional methods of genotyping are too slow and expensive to allow this new data to be applied on a large scale.

High throughput methods have been developed for large scale SNP scoring based on single base extension (SBE) of oligonucleotide primers using arrays of fluorescently labeled microspheres. Such systems provide accurate genotyping in a flexible format with ten-fold higher throughput and ten-fold lower costs than conventional genotyping methods. For example, U.S. Pat. No. 5,981,180 by Chandler et al. describes a method for the multiplexed diagnostic and genetic analysis of enzymes, DNA fragments, antibodies and other biomolecules. In their method, an appropriately labeled beadset is constructed, the beadset is exposed to a clinical sample, and the combined beadset/sample is analyzed by flow cytometry. Their method employs a pool of beadsets wherein beads within a subset differ in at least one distinguishing characteristic from beads in any other beadset. In that manner, the subset to which a bead belongs can be readily determined after beads from different subsets are combined. The distinguishing characteristics between beadsets are provided by incorporation of two or more fluorophores into the beads. Given suitable fluorophores and detection equipment, use of multiple fluorophores could expand the multiplexing power of the system.

However, the multiplexed analysis capacity of typical fluorescent microsphere arrays is currently limited to one hundred simultaneous assays, and expansion beyond this number involves a number of technical challenges. In addition, the routine preparation of these fluorescent microspheres still presents problems.

Solid phase arrays have also been used for the rapid and specific detection of multiple polymorphic nucleotides. Typically, an allele-specific hybridization probe is linked to a solid support and a target nucleic acid (e.g., a genomic nucleic acid, an amplicon, or, most commonly, an amplified mixture) is hybridized to the probe. Either the probe, or the target, or both, can be labeled, typically with a fluorophore. Where the target is labeled, hybridization is detected by detecting bound fluorescence. Where the probe is labeled, hybridization is typically detected by quenching of the label. Where both the probe and the target are labeled, detection of hybridization is typically performed through monitoring of a color shift resulting from proximity of the two bound labels. A variety of labeling strategies, labels, and the like, particularly for fluorescent based applications are described.

In one embodiment, an array of probes is synthesized on a solid support. Exemplary solid supports include glass, plastics, polymers, metals, metalloids, ceramics, organics, and the like. Using chip masking technologies and photoprotective chemistry it is possible to generate ordered arrays of nucleic acid probes. These arrays, which are known, e.g., as “DNA chips,” or as very large scale immobilized polymer arrays (VLSIPS™ arrays) can include millions of defined probe regions on a substrate having an area of about 1 cm² to several cm², thereby incorporating sets of from a few to millions of probes. The construction and use of solid phase nucleic acid arrays to detect target nucleic acids is well described in the literature. See, e.g., Fodor et al. (1991) Science, 251: 767-777; Hubbell U.S. Pat. No. 5,571,639; and, Pinkel et al. PCT/US95/16155 (WO 96/17958).

Magnetic particles made from magnetite and inert matrix materials have long been used in the field of biochemistry. Such particles generally range in size from a few nanometers up to a few microns in diameter and may contain from 15% to 100% magnetite. They are often described as superparamagnetic particles or, in the larger size range, as beads. The usual methodology is to coat the surface of the particles with some biologically active material that will cause them to bond strongly with specific microscopic objects or particles of interest (e.g., proteins, viruses, and DNA fragments). The particles then become “handles” by which the objects can be moved or immobilized using a magnetic gradient, usually provided by a strong permanent magnet. U.S. Pat. No. 4,537,861 by Elings et al. describes an example of tagging using magnetic particles. Specially constructed fixtures using rare-earth magnets and iron pole pieces are commercially available for this purpose. However, in this process, magnetic particles are never used in labeled subsets of particles allowing for a multiplexed assay of a sample.

In another approach using magnetic particles, U.S. Pat. No. 5,252,493 by Fujiwkara et al. describes a ultra-sensitive laser magnetic immunoassay method including: labeling an antigen or antibody with micro-particles of a magnetic substance to form a magnetic-labeled body; subjecting a specimen and the magnetic-labeled body to an antigen-antibody reaction to form a reacted body-specimen complex; separating and removing unreacted body from the reacted complex; guiding and concentrating the reacted complex magnetically; irradiating the concentrated complex with a laser beam; detecting outgoing light from a measurement system to provide a quantitative result in the picogram range. Again in this process, magnetic particles are never used in labeled subsets of particles allowing for a multiplexed assay of a sample.

It would be beneficial if another method were available for detecting the presence of a sought-after, predetermined target, e.g., such as a nucleotide sequence or allelic variants. It would further be beneficial if such a detection method were capable of providing multiple analyses in a single assay (multiplex assays).

### SUMMARY OF THE INVENTION

In accordance with the purposes of the present invention, as embodied and broadly described herein, the present invention includes a particle identification apparatus including a flow cell for passage of fluid containing a population of labeled magnetic microspheres in a stream, the magnetic microspheres having a label providing a detectable property to the magnetic microspheres, and, a magnetic measurement system, positioned adjacent to the flow cell, for measuring a magnetic moment on each labeled magnetic microsphere as it passes by the magnetic measurement system. In one embodiment of this particle identification apparatus, the apparatus further includes a detection system for measuring a detectable property from each labeled magnetic microsphere.

The present invention further includes a particle sorting apparatus including a chamber having an inlet for a fluid suspension of a population of magnetic mictospheres to be sorted, a magnetic field generator that produces a field gradient across the chamber for producing a force on the magnetic microspheres within the fluid suspension, a series of collection bins positioned within the chamber for receiving magnetic microspheres with distinctly different magnetic moments as a result of movement of the magnetic microspheres resulting from force produced on the magnetic microspheres within the fluid suspension by magnetic field gradient; and, an outlet for fluid flow.

The present invention further includes a kit for sorting and identifying a target material within a sample, the kit including a population of magnetic microspheres each having a distinctly measurable magnetic moment, with each magnetic microsphere also having one or more receptor agents attached thereto; and, a population of non-magnetic microspheres, with each magnetic microsphere also having one or more receptor agents attached thereto.

The present invention further includes a kit for sorting and identifying a target material within a sample, the kit including at least two populations of magnetic microspheres each population having a distinctly different measurable magnetic moment. In another embodiment of this kit, magnetic microspheres within each of the at least two populations also have one or more receptor agents attached thereto.

### BRIEF DESCRIPTION OF THE DRAWINGS

FIG. 1 shows a schematic drawing of a particle sorting apparatus for sorting of magnetic particles or magnetic microspheres by magnetic moment, M, in accordance with the present invention.

FIG. 2 shows a schematic drawing of an apparatus for single particle based analysis and analytical scale sorting in accordance with the present invention.

### DETAILED DESCRIPTION

The present invention is concerned with: 1) a unique particle sorting apparatus or system for fractionation of magnetic particles or magnetic microspheres; 2) an apparatus or system for performing magnetization measurements in a flow cytometry-like system for assay of qualitative and/or quantitative magnetic characteristics of a sample; 3) an apparatus or system for performing substantially simultaneous magnetization measurements and a second measurable property such as, e.g., fluorescence, in a flow cytometry-like system for assay of qualitative and/or quantitative characteristics of a sample; 4) kits including at least two distinct populations of microspheres for sorting and identifying a material within a sample, one population of magnetic microspheres, and, one population of non-magnetic microspheres, the microspheres also having one or more receptor agents attached thereto; and 5) kits including at least two distinct populations of magnetic microspheres for sorting and identifying a material within a sample, where each population of magnetic microspheres has a distinctly different measurable magnetic moment.

The apparatus and kits of this invention can be used in practicing the processes described by Kraus, Jr. et al., in U.S. patent application Ser. No. xx/xxx,xxx, filed concurrently herewith, entitled “Bioassay and Biomolecular Identification, Sorting and Collection Methods using Magnetic Microspheres”, such description incorporated herein by reference.

Within the present description, the term “microparticle” is meant to refer to small particles, generally from about 0.01 microns to about 1000 microns, that include an inherent property (e.g., magnetization, fluorescence and the like) allowing identification of each microparticle as belonging to a specific group. The term “microsphere” is meant to refer to a particle within the range of from about 0.01 microns to about 1000 microns consisting of one or more identifying tags (e.g., magnetization, fluorescence and the like) formed together with a polymer, glass, or other matrix, coating or the like. The term “magnetic microsphere” is meant to refer to a particle within the range of from about 0.01 microns to about 1000 microns including one or more magnetic domains with a polymer, glass, or other matrix, coating or the like. Neither the term “microsphere” or “magnetic microsphere” is meant to exclude shapes other than spherical, and such terms are meant to include other shapes such as globular and the like. The term “receptor” is meant to refer to a molecule or molecular fragment that is bound or otherwise attached to the surface of a microsphere.

Magnetic microsphere arrays and the instrumentation to analyze such arrays represent a major breakthrough in large-scale genomic analysis. Magnetic microsphere arrays consisting of a thousand elements (and potentially many more) can be prepared, and a flow cytometer can measure both fluorescence (e.g., by standard known techniques) and magnetization (using, e.g., Superconducting Quantum Interference Device (SQUID) technology or other magnetic detection technology) to provide greatly enhanced analysis capabilities. In essence, the magnetic microsphere array technology of the present invention serves as a bar-coding system to provide a detectable label to each microsphere.

In addition to different magnetic properties, i.e., magnetic moments, the microspheres in the present invention can have an additional detectable property. Such a detectable property can be, e.g., fluorescence, absorbance, reflectance, scattering and the like. Analytical equipment is commercially available to detect and determine each of these type properties. For example, flow cytometry systems are available employing laser based systems for detection and measurement of the fluorescence of a microsphere as it passes the detector.

The apparatus and kits of the present invention involve use of magnetic microspheres incorporating magnetic particles to “label” different assay agents. Such magnetic magnetic microspheres can be uniquely identified by their magnetic moment, M, by either measuring the moment directly or using a “spectrometer-like” apparatus described below. It is estimated that for magnetic microspheres of a size from about 10 nanometers (nm) to about 5 millimeters (mm), a system can be devised with between 10² and 10⁴ separable bins. The apparatus and kits of the present invention involve: magnetic microsphere preparation; an apparatus or system for separation of magnetic microspheres into populations with discrete magnetizations; attachment of receptor agents to microspheres; and, an apparatus or system for analysis of magnetic microspheres and quantitative measurement of additional measurable properties, e.g., fluorescence.

Magnetic particles can be obtained from a large variety of vendors. Investigating a relatively small number (much less than 100) of different simultaneous receptor agents can tolerate some variety of particle size and shape without interfering with the performance of the sorting process. Investigating large numbers of receptor agents simultaneously will require a greater number of particles, and greater uniformity of particle size and shape to prevent variations in drag from interfering in the sorting process. This can be readily accomplished by encapsulating the magnetic particles inside a glass or polymer coating to form microspheres or beads of uniform size. For example, magnetic particles can be encapsulated in polystyrene and provide a suitable surface for subsequent attachment of receptor agents. Encapsulation in uniformly sized and spherical glass beads is a commercially available technology. Although not required, magnetic particles preferably will initially have little or no magnetization (remnant field), and thus have very little dipole moment or mutual attraction. Any coating, imbedding, immobilizing or encapsulation of the magnetic particles (except for specific receptor agents) into a magnetic microsphere is preferably conducted prior to the magnetic particle sorting. After formation by coating, imbedding, immobilizing or encapsulation, magnetic particles are thereafter generally referred to as magnetic microspheres. In addition to encapsulation or coating for forming magnetic microspheres, the magnetic microspheres can be formed by imbedding magnetic particles within or on a surface of an organic polymer material or glass. The magnetic particles may protrude beyond the surface of the organic polymer material or glass. Similarly, the magnetic microspheres can be formed by immobilizing magnetic particles within or on a surface of an organic polymer material or glass. The magnetic particles may protrude beyond the surface of the organic polymer material or glass. Another manner of forming magnetic microspheres can involve coating magnetic particles with a material having first reactive functionality and reacting the coated magnetic particles with non-magnetic microspheres having a second reactive functionality that reacts with the first reactive functionality to form microspheres having the required magnetic component. Among suitable reactive functionalities are included functionalities, e.g., from the group of amines, carboxylates, and epoxies. Additionally, affinity pairs such as biotin-avidin and the like that form a complex can be used as the first and second reactive frinctionalities. Thus, both covalent binding and non-covalent interaction approaches may be employed.

Typically, the magnetic particles used in forming the magnetic microspheres will be of a size between about 1000 microns (μm) and 10 nanometers, although both smaller and larger particles may be used in some instances. The lower limit in size is likely about 100 nanometers (nm), although the required size may vary depending upon the chemical composition of the magnetic particles. In general, bigger particle sizes can be preferred as they provide greater surface area and volume for binding capacity. This can provide greater variety in magnetization through volume of the particles. In one embodiment for practicing the present invention, the utilized magnetic microspheres are preferably of the same dimensions to minimize fluid dynamic effects during subsequent sorting steps.

The suitable chemical compositions for the magnetic particles are generally ferromagnetic materials and include rare earth containing materials such as, e.g., iron-cobalt, iron-platinum, samarium-cobalt, neodynium-iron-boride, and the like. Other magnetic materials, e.g., superparamagnetic materials such as iron oxides (Fe₃O₄) may be used as well. Among the preferred magnetic materials are included iron-cobalt as such material is generally easier to magnetize, has a stronger magnetization (about 1.7 Tesla) and is less susceptible to corrosion.

The range in magnetic moment of the magnetic particles is generally 10⁻²² and 10⁻¹⁵ T-m³, more preferably above 10⁻²¹ T-m³. This magnetic moment is essentially maintained after formation of the magnetic microspheres as the coating does not affect the magnetic moment detectable by SQUID technology or other magnetic detection technologies. Magnetic moments of particles within the range indicated above are easily detected and measured by commercially available SQUID sensors at sub-millimeter to a few millimeter sensor-to-particle separations.

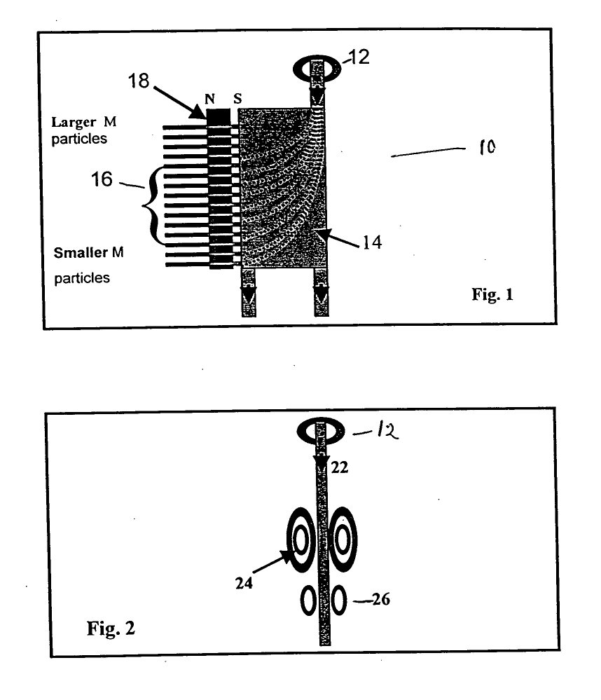

In one apparatus of the present invention, the magnetic particles, preferably as magnetic microspheres after coating, can be initially separated into populations with discrete magnetizations. A fluid suspension of the magnetic microspheres is introduced into the apparatus schematically shown in FIG. 1. This magnetic field particle fractionation flow device 10 enables the sorting of magnetic microspheres according to their magnetization. The suspended microspheres pass through magnetization coils 12 to magnetize the microspheres which then flow into the spectrometer chamber 14 where they follow trajectories determined largely by the magnetic moment, M, of each microsphere under the influence of, e.g., magnet 18 (one manner of providing the necessary magnetic field gradient), and are collected in magnetic microsphere collection bins 16. Such collection bins could be tubes. System performance parameters such as the number of different collection bins, sorting resolution, and signal-to-noise between adjacent bins are variable over orders of magnitude depending on system size, engineering tolerances, and the like. Preferably, there is a high resolution between magnetic microspheres with differing magnetic moments. Optionally, magnetic microsphere flow can be terminated and magnetic microspheres in each bin are collected after sorting the desired number of magnetic microspheres. Magnetic microspheres are captured within the collection bins by the field from the spectrometer magnetic field and do not rely upon the flow to maintain the separation.

The peak field of the magnetizing magnet is chosen to assure saturation magnetization is reached for the specific magnetic particles being used (e.g., the saturation field for Sm-Co is significantly greater than that for magnetite). Although a number of different magnetization systems can be employed, a suitable example is a series of “thin” pulsed solenoid magnets. The magnitude and duration of the solenoid pulse would be tuned to the specific type of magnetic material being used for the particles. The field profile of neighboring solenoids would overlap to assure all particles are magnetized. This is easily assured since the typical time to assure magnetization is on the order of a millisecond or less. The series of solenoids would be activated from the most upstream magnet. The next downstream magnet will be activated at time t=T_{M} before the upstream magnet is deactivated, where T_{M} is the magnetization time for the given magnetic particles. This algorithm assures all particles are magnetized. Since the distance traveled by the particles in the fluid flow during the time required magnetizing them, D_{M}, is far smaller than the solenoid field length, only two or three solenoids will be required in the series. After the last (downstream) solenoid is activated, the first (upstream) solenoid is activated again after a delay equal to the transit time of particles through the entire solenoid series field less 2(T_{S}), where T_{S} is the time required to activate all the magnets in the solenoid series as described above. Thus the delay is:

Activation Delay=L_{S}/V_{F}−(2T_{S})

where L_{S} is the total length of the solenoid series field (above the particle magnetization threshold), and V_{F} is the fluid flow velocity. This fairly complex magnetization method is designed to prevent the magnetic particles from being attracted by a strong and constant field gradient that would necessarily result from the magnetizing magnets. In systems and situations where there is a sufficient flow rate to prevent the magnetic particles from being attracted out of the flow, a single pulsed or continuous magnetizing field would be far simpler to implement.

Magnetic shielding should be provided between the magnetic microsphere spectrometer chamber and the flow tube. Such shielding will eliminate the possibility of the magnetization fields from the solenoid from interfering with particle motion in the spectrometer chamber and to prevent fields from the spectrometer magnetic from interfering with particle motion prior to entering the spectrometer chamber.

The magnetized microspheres experience a force within the spectrometer chamber proportional to the particle magnetic moment, M, and move along a trajectory that is determined by the magnetic force on the particle, the fluid flow, and drag. Techniques used in flow cytometry are employed to generate a uniform fluid flow throughout the chamber. Uniform flow within the spectrometer chamber is more important when the goal is identification and assay instead of sort and assay, both described below. In the application where the spectrometer is used to sort the magnetic microspheres a second time after combining a target sample with the number of distinct populations of magnetic microspheres containing the different attached receptor agents together for a period of time sufficient for binding between attached receptor agents and target species within the target sample to form one or more receptor agent-target species complexes, the requirement of uniform flow can be relaxed provided that flow through the chamber is reproducible in successive applications of the sorting step. Any effects of a non-uniform flow will cancel out between the first and second sorting steps.

Magnetic microspheres are collected in tubes (see FIG. 1) and captured by the field of the spectrometer magnet. The separation (shown in the figure) between collection tubes is exaggerated for clarity. The number and size in the separation system preferably has collection tubes adjoining one another with minimal wall thickness. In one embodiment, walls between the orifices to the collection tubes are tapered to knife-edges helping prevent magnetic microspheres from collecting on the ends of the walls. Flow through the collection tubes would further facilitate keeping magnetic microspheres from accumulating at the orifices of the tubes.

There are numerous engineering requirements and design variables involved for design of this system including the fluid flow velocity, range of particle magnetic moments, range of particle sizes, desired size of the apparatus, numbers of bins desired (system resolution), particle throughput for the system, and the like. Such variables are well known by those skilled in the art.

In one embodiment, the magnetic microspheres are demagnetized prior to proceeding on to the next step. This can be readily accomplished by heating above the material Curie point as is well known to those skilled in the art. Demagnitized microspheres are easier to work with because there is significantly less tendency to aglomerate. Alternatively, the magnetic microspheres remain magnetized while proceeding on to the next step. Experience has shown that any agglomerated microspheres can be easily separated by several simple methods such as ultrasonication of the samples.

Attachment of the receptor agents to the microspheres can be conducted in the following manner. The process of attaching receptor agents is generally the same for magnetic microspheres or non-magnetic microspheres. Once magnetic microspheres are “sorted,” the bins that are collected are maintained separately. A different receptor agent (or assay agent) can attached to magnetic microspheres from each retained bin. Once the receptor is attached, an investigator can simply choose the amount of each receptor agent (attached to different magnetic microsphere groups) to introduce into the medium being investigated. For purposes of genotyping applications, the receptor is preferably a synthetic oligonucleotide of DNA covalently attached to the magnetic microsphere surface. The receptor agent is generally of, e.g., nucleic acids such as, e.g., DNA, RNA and nucleotides, proteins such as, e.g., antibodies, antigens and peptides, lipids, carbohydrates, synthetic polymers or any other specific receptor molecules. The receptor agent is generally specific in binding to a particular target species or class of target species. The receptor agent may be attached to the magnetic microsphere surface by various methods including, but not limited to, physical adsorption, specific binding, or chemical conjugation.

The apparatus and kits provided by the present invention find use in a wide number of applications. A number of potential applications follow and other applications will be readily apparent to those skilled in the art. For example, in one embodiment in accordance with the present invention, analysis of target samples can be conducted. In another embodiment in accordance with the present invention, sorting of target samples can be conducted.

Analysis may be carried out on biological systems or other samples such as chemical systems. For analysis of biological systems, a receptor agent can be, e.g., an immobilized molecule of DNA, including cDNA and oligonucleotides, an immobilized molecule of RNA, an immobilized protein (including an antibody, an antigen and a peptide), an immobilized lipid, an immobilized carbohydrate, an immobilized sugar or an immobilized synthetic polymer. Use of an immobilized molecule of DNA as receptor agents can allow specific assays, e.g., for single nucleotide polymorphisms (SNPs), for sequencing validation, for genotyping, for bacteria identification, for DNA-based tissue typing, for multiplexed viral load analysis, for gene expression, for DNA-protein interaction, and for molecular assembly. Use of an immobilized molecule of RNA as receptor agents can allow specific assays, e.g., for RNA-protein interaction, and for molecular assembly. Use of an immobilized protein molecule as the receptor agent can allow specific assay, e.g., for protein expression, immunoassay, immunoprecipitation, biomarker discovery, protein-protein interaction, for protein-DNA interactions, for protein-RNA interactions, or for protein-other molecule interactions; for antibody-antigen interactions. Use of an immobilized lipid as the receptor agent can allow for a specific assay in the form of biosensors, for molecular assembly and for lipid-other molecule interaction.

Examples of potential immunoassay applications include but are not limited, e.g., to: detection of antibodies specific for both proteinase 3 (PR3) and myeloperoxidase (MPO), such detection important in the diagnosis of systemic vasculitis; an assay for simultaneous detection of serum IgG responses to Toxoplasma gondii, rubella virus, cytomegalovirus, and herpes simplex virus types 1 and 2; an assay for the simultaneous multiplexed assay quantifying human serum IgG1, IgG2, IgG3, IgG4, IgA and IgM in a single tube; allergy testing; autoimmune testing; epitope-mapping; multiplexed analysis of human cytokines; a multiplexed array for measurement of human chorionic gonadotropin (hCG) and alpha-fetoprotein (aFP); and, pneumococcal assay to measure antibodies to the 23-serotype pneumococcal capsular polysaccharides (PPS).

Examples of potential gene expression or protein expression applications include but are not limited to, e.g., identification of distinctive expression patterns characteristic of selected physiological and pathological states, and screening for subtle changes in response to various stimuli or environmental change.

Examples of potential biomarker discovery include but are not limited to search for schizophrenia diagnostic markers, kidney stone disease markers, prostrate cancer markers, validation of protein markers and the like.

Examples of potential antibody-antigen interaction applications include but are not limited to, e.g., antigen capture and identification for antibodies or antibody capture and identification for antigens.

Examples of potential protein-protein interaction applications include but are not limited to, e.g., capture and purification of a potential ligand for use as a receptor agent, measurement accurate intact mass of a captured analyte, and identification of a captured analyte by “on-bead” peptide mapping.

In another application, the present invention may also allow for the sorting of multiple cells, protein, DNA fragments, RNA fragments and other molecules.

A standard flow cytometry system can be used to analyze the fluorescence from any particular microsphere. It is well known how to form a sequential flow steam of particles for use in a flow cytometer or similar sensitive fluorescence detection apparatus. See, e.g., U.S. Pat. No. 3,710,933 by Fulwyler et al. and Flow Cytometry and Sorting, 2nd Ed., ed. M. R. Melamed et al. Wiley-Liss, New York, 1990, incorporated herein by reference. Basically, a dilute solution of magnetic microspheres is formed to a low concentration effective to provide the microspheres spaced apart in the flow stream so that only a single microsphere is present in the excitation and detection volume. The solution of magnetic microspheres is then introduced into a laminar sheath flow stream for passage through the detection chamber for light excitation of a single microsphere at a time. The flow rates of the sample and the sheath can be adjusted to maintain separation between microspheres and to provide the optimum time for each microsphere in the excitation source.

In the present invention, the analytical instrumentation includes the standard flow cytometry parts for analysis of the fluorescence of the individual microspheres in combination with the necessary magnetic detection technology, e.g., SQUIDS, for measurement of the magnetic characteristics of the individual microspheres. SQUIDs can be positioned in close proximity to the chamber for fluorescence measurement such that substantially simultaneous fluorescence and magnetization measurements can be performed. By “substantially simultaneous” it is meant to indicate that the correlation between magnetic and fluorescence or other detectable property measurements for any given particle is unambiguous with respect to neighboring particles in the flow stream. That is, passage of an individual magnetic microsphere past SQUID sensors can allow for identification, through detection of the magnetic moment, of a particular subset of microspheres to which each individual microsphere belongs. That can be coupled with the standard fluorescence measurements from the receptor agents on each subset of magnetic microspheres. In this manner the present invention can allow for use of the flow cytometer and magnetic microsphere arrays to perform multiplexed genetic analysis.

In the practice of the present invention, a mixture of magnetic microspheres with attached receptor agents is combined with the medium being investigated and incubated as appropriate. In some cases the medium is treated with some combination of reagents to generate a fluorescent complex that can be captured onto the magnetic microspheres. Such reagents may include an antibody or other ligand, an oligonucleotide or analogue, or other molecule with specific binding or enzymatic activity. In other cases, the receptors attached to the magnetic microspheres are themselves fluorescent and report the presence of analyte as a change in fluorescence. For the genotyping applications, a DNA polymerase-mediated extension of oligonucleotide primers with fluorescent nucleotide analogues can be used that have successfully performed on fluorescent microspheres.

The incubation media is prepared for use in one of the two flow systems used to perform the final particle identification and fluorescent measurements. Two methods for sorting the magnetic microspheres and assaying the efficiency and/or effectiveness of the agents of interest are described below.

In one embodiment of the present invention, the method simultaneously identifies magnetic microspheres based on magnetic moment and measures another detectable property, e.g., the fluorescence, associated with each magnetic microsphere. By “identification”, it is meant that a particular magnetic microsphere can be associated to belong to a specific sorted group based on the magnetic moment of the particle and accordingly be known to have a particular magnetic label. It is important to note that an absolute measurement of the magnetic moment of the microsphere is not required, rather a relative measurement with sufficiently high resolution to uniquely associate the particle with a specific sorted group of microspheres. A schematic of this method is shown in FIG. 2. A suspension of magnetic microspheres is introduced into a sheath stream 22 that hydrodynamically focuses the sample stream in a flow cell. The magnetic moment, M, of the magnetized microspheres is measured by a SQUID array 24 as individual microspheres flow past the sensors (preferably at least two). In one embodiment, a microsphere-aligning field can be used that orients the particles passing under the SQUID, but does not couple to the SQUID (e.g., the field is tangential to the SQUID). In one embodiment, at least two SQUID sensors can be required, one on either side of the flow channel to correct for microsphere position within the flow cell. Small High-T_{c} SQUID sensors will be used for this purpose with a dewar configured to minimize the SQUID-flow cell distance, similar to the SQUID microscope described by Epsy et al., in IEEE Trans. Appl. Superconductivity, v. 9, p. 3692 (1999). Minimizing the SQUID-flow channel distance will improve the sensitivity of the sensor to microsphere field, and reduce the relative magnitude of superposed field from neighboring microspheres (e.g., error). The typical microsphere separation in flow systems currently in use is on the order of centimeters, more than enough to prevent any appreciable superposition of field from neighboring microspheres. If needed, moment corrections can easily be calculated on the basis of moment and proximity of first and even second nearest neighbors. Typical fields of the magnetic microspheres at the SQUID sensors are expected to be easily detectable 1-100,000 picoTesla (pT) for 1-5 micron (μm) magnetic microspheres of high remnant field materials assuming 3 mm sensor-microsphere separation (readily attainable). After the relative measurement of M, the fluid carries the magnetic microsphere past a laser/sensor apparatus 26 to perform the fluorescence measurement. Several different fluorescence emission signals may be collected from each microsphere. Analysis of the correlated magnetization and fluorescence data can then be performed. As with conventional fluorescence-activated cell sorting, single magnetic microsphere measurements can be used to control the physical sorting of individual magnetic microspheres. While this approach to sorting enables very specific criteria to be applied to sort decisions, it is generally restricted to sorting two to four populations and is fairly low throughput.

In another embodiment of the present invention, the method utilizes the same sorting method to separate the magnetic microspheres into bins as was implemented in Step 2. After incubation, the suspension of magnetic microspheres is introduced into the apparatus shown in FIG. 1. This method has at least three significant advantages over the first described embodiment: 1) magnetic microspheres can be sorted at a much higher rate; 2) a much broader range of magnetic microspheres can be used; and 3) systematic errors in the sorting (e.g. flow uniformity, particle size and shape, etc.) will largely cancel. The identification method measures the relative particle |M| requiring well-separated magnetic microspheres. While not problematic for applications that require only particle analysis, these constraints represent significant limitations for preparative sorting applications. The spectrometer sorting method is capable of sorting a fluid flow containing a particle density that is orders of magnitude larger than in the identification scheme, limited only the requirement that neighboring magnetic microspheres do not impart an appreciable force on one another.

Once the magnetic microspheres are sorted into their respective bins, the quantitative fluorescence assay is performed using the established methods. The suspended particles flow past a laser/detector system that identifies the desired bound states. Two basic system approaches can be developed: a two-stage method or a single-stage continuous flow process. Alternately, the analyte bound to the receptor on the magnetic microspheres can be analyzed off-line by some other analytical method (mass spectrometry, NMR, and the like). The ability to perform preparative-scale purification of biomolecules is an especially important feature of this second sorting method.

A new approach in performing assay experiments, e.g., bioassay experiments, is presented by this invention. The basic approach is to use magnetic microspheres as addressable solid supports in bioassay experiments. Magnetic microspheres from 100 nm to 10 μm in size or more can be obtained with a continuous range of magnetic moments from near zero to upper limits that depend on the material (or materials) chosen. The number of separation bins that could partition the magnetic microspheres is determined by engineering considerations, but ranges from a low range of about 100 to up to a high range of about 1 million. For very large numbers of bins, two-stage sorting is preferable as described above to be practical. The ability to address large numbers of solid supports, each of which bears a different receptor, would enable the performance of highly parallel multiplexed analysis of many analytes simultaneously. This method can be adapted to both high throughput assay analysis systems such as that described above, or extremely high throughput physical sorting devices as also described above. The present invention provides an instrument platform to enable many types of genomic and biochemical analysis on a scale not currently possible.

The variation in size and shape will be small enough to allow at least several subpopulations of magnetic microspheres to be identified. For very highly multiplexed applications, uniform spherical glass beads or polymer beads containing magnetic particles may have less variation in size and shape, allowing more discrete populations to be identified.

The principle of this magnetic microsphere separation technique is similar to that of continuous flow electrophoresis, a widely used preparative separation method, except that the electric field is replaced with a magnetic field. Instrumentation for continuous flow separation are described in the literature (see, e.g., J. Chromatography B 722:121-139 (1999)) and are commercially available. Modification of such designs to enable magnetic separation is well within the capabilities of one skilled in the art.

Immobilization of biomolecules as receptors on microsphere surfaces is well known to those skilled in the art. There are several conjugation chemistries available for the attachment of synthetic DNA to the surface of polystyrene or glass surfaces.

Several assay systems have been developed involving microspheres and flow cytometry, including the detection of DNA from pathogenic bacteria and the identification of genes associated with chronic beryllium disease. Such systems can be used.

The sensitivities of the fluorescence detection and SQUID sensing are well within the range required for the measurement of single magnetic microspheres. The main hurdles will be engineering issues associated with the integration of a magnetically shielded SQUID sensor in close proximity to the laser light source and light collection optics to enable dual magnetization and fluorescence measurements to be made on single magnetic microspheres in the flowing sample stream of the flow cytometer.

A number of kits are provided by the present invention. Such kits include more than one different population of microspheres and such kits find use in sorting and identifying materials within a sample. In one embodiment of such a kit, the kit includes a first population of magnetic microspheres each having a distinctly measurable magnetic moment, with each individual magnetic microsphere also having one or more receptor agents attached thereto; and, a second population of non-magnetic microspheres, with each individual non-magnetic microsphere also having one or more receptor agents attached thereto. Each population provides two separate detectable properties.

In another embodiment of such a kit, the kit includes at least two populations of magnetic microspheres each population having a distinctly different measurable magnetic moment. In this embodiment, the different populations of magnetic microspheres can each have one or more receptor agents attached thereto as well to provide two separate detectable properties.

Although the present invention has been described with reference to specific details, it is not intended that such details should be regarded as limitations upon the scope of the invention, except as and to the extent that they are included in the accompanying claims.

1-14. (cancelled).

15. A particle sorting apparatus comprising:
a chamber having an inlet for a fluid suspension of a population of magnetic microspheres to be sorted;
a magnetic field generator that produces a field gradient across said chamber for producing a force on said magnetic microspheres within said fluid suspension;
a series of collection bins positioned within the chamber for receiving magnetic microspheres with distinctly different magnetic moments as a result of movement of said magnetic microspheres resulting from the force produced on said magnetic microspheres within said fluid suspension by magnetic field gradient; and,
an outlet for fluid flow.

16. The particle sorting apparatus of claim 15 further including a means of magnetizing said population of magnetic microspheres prior to entry into said chamber.

17. The particle sorting apparatus of claim 15 wherein said means for magnetizing is a magnet having a peak field greater than the saturation magnetization of the magnetic microspheres.

18. The particle sorting apparatus of claim 15 wherein said magnetic field generator is selected from the group consisting of a magnet and a magnetic field coil positioned at a side of said chamber.

19. A kit for sorting and identifying a material within a sample, the kit comprising:
a population of magnetic microspheres each having a distinctly measurable magnetic moment, with each individual magnetic microsphere also having one or more receptor agents attached thereto; and,
a population of non-magnetic microspheres, with each individual non-magnetic microsphere also having one or more receptor agents attached thereto.

20. The kit of claim 19 wherein said receptor agents further include a detectable property label thereon.

21. The kit of claim 20 wherein said detectable property label is selected from the group consisting of fluorescence, absorbance, reflectance and scattering.

22. The kit of claim 20 wherein said detectable property label is fluorescence.

23. The kit of claim 19 wherein said receptor agents further include a detectable labeled target analogue thereon.

24. The kit of claim 23 wherein said target analogue is selected from the group consisting of antigens, antibodies, peptides, proteins, nucleic acids, lipids, carbohydrates and enzymes.

25. The process of claim 19 wherein said magnetic microspheres include magnetic particles of a material selected from the group consisting of a ferromagnetic material and a superparamagnetic material.

26. The kit of claim 19 wherein said magnetic microspheres include magnetic particles of a material selected from the group consisting of iron-cobalt, iron-platinum, and samarium-cobalt.

27. The kit of claim 19 wherein said one or more receptor agents are for a target species selected from the group consisting of antigens, antibodies, peptides, proteins, nucleic acids, lipids, carbohydrates and enzymes.

28. The kit of claim 19 wherein said magnetic microspheres include magnetic particles coated with a coating material selected from the group consisting of an organic polymeric material and glass.

29. The kit of claim 19 wherein said magnetic microspheres include magnetic particles imbedded within a material selected from the group consisting of an organic polymeric material and glass.

30. The kit of claim 19 wherein said magnetic microspheres include magnetic particles immobilized on a surface of or within a material selected from the group consisting of an organic polymeric material and glass.

31. The kit of claim 19 wherein said magnetic microspheres include the reaction product of magnetic particles coated with a material having a first reactive functionality; and, non-magnetic microspheres having a second reactive functionality, said second reactive functionality adapted for reaction with said first reactive functionality.

32. The process of claim 31 wherein said first reactive functionality is selected from the group consisting of amines, carboxylates, epoxies and one of an affinity pair, and said second reactive functionality is different from said first reactive functionality and is selected from the group consisting of amines, carboxylates, epoxies, and the other of the affinity pair.

33. A kit for sorting and identifying a material within a sample, the kit comprising at least two populations of magnetic microspheres each population having a distinctly different measurable magnetic moment.

34. The kit of claim 33 wherein magnetic microspheres within each of said at least two populations also have one or more receptor agents attached thereto.

35. The process of claim 33 wherein said magnetic microspheres include magnetic particles of a material selected from the group consisting of a ferromagnetic material and a superparamagnetic material.

36. The kit of claim 33 wherein said magnetic microspheres include magnetic particles of a material selected from the group consisting of iron-cobalt, iron-platinum, and samarium-cobalt.

37. The kit of claim 34 wherein said one or more receptor agents are for a target species selected from the group consisting of antigens, antibodies, peptides, proteins, nucleic acids, lipids, carbohydrates and enzymes.

38. The kit of claim 33 wherein said magnetic microspheres include magnetic particles coated with a coating material selected from the group consisting of an organic polymeric material and glass.

39. The kit of claim 33 wherein said magnetic microspheres include magnetic particles imbedded within a material selected from the group consisting of an organic polymeric material and glass.

40. The kit of claim 33 wherein said magnetic microspheres include magnetic particles immobilized on a surface of or within a material selected from the group consisting of an organic polymeric material and glass.

41. The kit of claim 33 wherein said magnetic microspheres include the reaction product of magnetic particles coated with a material having a first reactive functionality; and, non-magnetic microspheres having a second reactive functionality, said second reactive functionality adapted for reaction with said first reactive functionality.

42. The process of claim 41 wherein said first reactive functionality is selected from the group consisting of amines, carboxylates, epoxies and one of an affinity pair, and said second reactive functionality is different from said first reactive functionality and is selected from the group consisting of amines, carboxylates, epoxies, and the other of the affinity pair.
